(19) United States
(12) Patent Application Publication
(10) Pub. No.: US 2005/0163839 A1
(43) Pub. Date: Jul. 28, 2005

(54) Oral controlled release pharmaceutical composition containing metaxalone as active agent

(75) Inventors: Kamlesh Mohanlal Dudhara, Baroda (IN); Nitin Bhalachandra, Dharmadhikari (IN); Yashoraj Rupsinh, Zala (IN)

Correspondence Address: MERCHANT & GOULD PC, P.O. BOX 2903, MINNEAPOLIS, MN, 55402-0903, US

(73) Assignee: SUN PHARMACEUTICAL INDUSTRIES LIMITED, Mumbai (IN)

(21) Appl. No.: 10/502,896

(22) PCT Filed: Jan. 29, 2003
(86) PCT No.: PCT/IN03/00014

(51) Int. Cl. A61K 9/22

(52) U.S. Cl. 424/468

(57) ABSTRACT

The present invention provides an oral controlled release pharmaceutical composition comprising metaxalone, a pharmaceutically acceptable release rate controlling excipient, and pharmaceutically acceptable excipients, wherein the oral controlled release pharmaceutical composition provides peak plasma levels at a time of about 3 hours or more after oral administration of the composition.

### FIELD OF THE INVENTION

The present invention provides an oral controlled release pharmaceutical composition for metaxalone.

### BACKGROUND OF THE INVENTION

Metaxalone, [5-(3,5-Dimethylphenoxymethyl)-2-oxazolidinone] disclosed in the U.S. Pat. No. 3,062,827 is indicated as an adjunct to rest, physical therapy, and other measures for the relief of discomforts associated with acute, painful musculoskeletal conditions. The mode of action of metaxalone has not been clearly indicated, but may be related to its sedative properties. Metaxalone does not directly relax tense muscles in man. The recommended dose of metaxalone for adults and children over 12 years of age is two tablets (800 mg) three to four times daily.

Controlled release drug delivery systems deliver drug to the body so as to establish therapeutically effective blood levels of the active ingredient and once these blood levels are achieved they continue to maintain constant blood levels for long duration. By avoiding peaks and troughs in blood levels associated with conventional dosage forms, controlled release systems lower the incidence of adverse effects or side effects. Very importantly controlled drug delivery systems reduce the frequency of dosing leading to convenience to the patient in terms of dosing and compliance to the specified dosage regimens. For these reasons an oral pharmaceutical composition of metaxalone would benefit therapy.

European Patent Application No. 147780 relates to compositions comprising therapeutic active agent containing core coated with super hydrolyzed polyvinyl alcohol for modifying the release rate of drugs. The disclosure would not enable one to design oral controlled release pharmaceutical composition for metaxalone that provided desirable plasma concentration. None of the examples demonstrated the rate and extent to which the release of a drug was controlled. No example of a metaxalone composition was provided. There was no mention of potential problems associated with controlling the release of a drug such as metaxalone. For example, metaxalone has a low aqueous solubility and prior known formulations show a lack of correlation between in vitro release and in vivo bioavailability. Further, it is not known whether metaxalone is absorbed throughout the gastrointestinal tract uniformly or only from the upper part of the gastrointestinal tract. There is thus a need for oral controlled release pharmaceutical composition that provides desirable plasma levels of metaxalone for twice-a-day or once-a-day therapy.

### OBJECT OF THE INVENTION

It is an object of the present invention to provide an oral controlled release pharmaceutical composition for metaxalone.

It is a further object of the invention to provide an oral controlled release pharmaceutical composition that provides a desirable plasma level of metaxalone for twice-a-day or once-a-day therapy.

### SUMMARY OF THE INVENTION

The present invention provides an oral controlled release pharmaceutical composition comprising metaxalone, a pharmaceutically acceptable release rate controlling excipient, and pharmaceutically acceptable excipients, wherein the oral controlled release pharmaceutical composition provides peak plasma levels at a time of about 3 hours or more after oral administration of the composition.

### BRIEF DESCRIPTION OF THE DRAWING

FIG. 1 shows the plasma concentration Vs time profile obtained upon administration of one embodiment of the oral controlled release pharmaceutical composition of the present invention comprising 400 mg metaxalone (Example 1).

### DETAILED DESCRIPTION OF THE INVENTION

The present invention provides an oral controlled release pharmaceutical composition comprising metaxalone, a pharmaceutically acceptable release rate controlling excipient, and pharmaceutically acceptable excipients, wherein the oral controlled release pharmaceutical composition provides peak plasma levels at a time of about 3 hours or more after oral administration of the composition.

The metaxalone that may be used in the present invention includes any solid particulate form of metaxalone. Crystalline metaxalone when used is preferably micronised. The solid particulate form of metaxalone of the present invention may have a reduced crystallinity or may be a crystal form of metaxalone with higher solubility in an aqueous medium than the known crystalline form of metaxalone. In yet another embodiment, the metaxalone used may be amorphous in nature. Metaxalone may also be used in the present invention as a solid admixture of metaxalone and a pharmaceutically acceptable excipient which admixture provides a higher intrinsic dissolution or solubility than the crystalline metaxalone in an aqueous medium. Metaxalone may also be used in the present invention in the form of a more soluble prodrug or derivative.

The pharmaceutical composition of the present invention may be in the form of a matrix formulation, a coated composition, an ion exchange composition, an osmotic system comprising a core covered with a semipermeable membrane, a gastric retention controlled drug delivery system, and various other controlled release compositions known to a person skilled in the art. A matrix formulation for the present invention comprises metaxalone and a release rate controlling excipient in admixture. A coated composition that provides a controlled release of metaxalone is obtained by forming a core comprising metaxalone and coating the core with a coating composition comprising release rate controlling excipients. The osmotic system for the controlled release of metaxalone comprises a core comprising metaxalone and other pharmaceutically acceptable excipients, covered with a semipermeable membrane, the membrane having an orifice for the release of metaxalone in a controlled manner over a defined period of time. In the osmotic system of the present invention, the release rate is determined by the osmotic pressure difference across both sides of the semipermeable membrane. Thus, the polymer comprising the semipermeable membrane may be considered the rate controlling excipient and core excipients influencing the osmotic pressure difference may also be considered the rate controlling excipients. A gastric retention controlled drug delivery system may be obtained by methods known to a person skilled in the art, preferably by using a release rate controlling excipient in admixture with a gas-generating agent.

One embodiment of the present invention may be a matrix formulation comprising release rate controlling excipients that may be prepared by mixing the active ingredient with a release rate controlling excipient. The release rate controlling excipient is any material that slows the rate of release of the drug from the dosage form. Usually, the release rate controlling excipient is a polymer or a fatty compound or a mixture thereof. It may also comprise an ion-exchange resin.

Examples of rate controlling polymers that may be used in the present invention, when in the form of a matrix system or a coated system, include but are not limited to:
cellulose ethers such as methylcellulose (MC), ethylcellulose (EC), hydroxyethylcellulose (HEC), hydroxypropyl cellulose (HPC), hydroxypropyl methylcellulose (HPMC), hydroxypropyl ethylcellulose (HPEC), carboxymethyl cellulose (CMC), crosslinked carboxymethyl cellulose (croscarmellose) and its alkali salts, ethylhydroxyethylcellulose (EHEC), hydroxyethyl methylcellulose (HEMC), hydrophobically modified hydroxyethyl cellulose (HMHEC), hydrophobically modified ethylhydroxyethylcellulose (HMEHEC), carboxymethyl hydroxyethylcellulose (CMHEC), carboxymethyl hydrophobically modified hydroxyethyl cellulose (CMHMHEC), and the like;
vinyl pyrrolidone polymers such as crosslinked polyvinylpyrrolidone or crospovidone, copolymers of vinyl pyrrolidone and vinyl acetate;
alkylene oxide homopolymers such as polypropylene oxide, preferably ethylene oxide homopolymers
a superdisintegrant polymer such as cross-linked polyvinylpyrrolidone, cross-linked sodium carboxymethylcellulose, carboxymethyl starch, sodium carboxymethyl starch, potassium methacrylate-divinylbenzene copolymer, polyvinyl alcohols, amylose, cross-linked amylose, starch derivatives, microcrystalline cellulose and cellulose derivatives, alpha-, beta-and gamma-cyclodextrin and dextrin derivatives such as cross-linked carboxymethylcellulose
gums of plant, animal, mineral or synthetic origin such as (i) agar, alginates, carrageenan, furcellaran derived from marine plants, (ii) guar gum, gum arabic, gum tragacanth, karaya gum, locust bean gum, pectin derived from terrestrial plants, (iii) microbial polysaccharides such as dextran, gellan gum, rhamsan gum, welan gum, xanthan gum, and (iv) synthetic or semi-synthetic gums such as propylene glycol alginate, hydroxypropyl guar and modified starches like sodium starch glycolate, and the like; and
an acrylic acid polymer such as cross-linked polymer available under the trade name Carbopol® or homopolymers and co-polymers of acrylate or methacrylate monomers for example polymethacrylates marketed under the brand names of Eudragit® particularly Eudragit® RS and Eudragit® RL.

Examples of fatty compounds that may be used as the release rate controlling excipients include various waxes such as digestible, long chain (C₈-C₅₀, especially C₁₂-C₄₀), substituted or unsubstituted hydrocarbons, such as fatty acids, fatty alcohols, glyceryl esters of fatty acids, mineral and vegetable oils and waxes. Hydrocarbons having a melting point of between 25° and 90° C. are preferred. Of these long chain hydrocarbon materials, fatty (aliphatic) alcohols are preferred.

The matrix formulation may also include various pharmaceutically acceptable excipients, for example wicking agents such as microcrystalline cellulose; disintegrants such as starch, cellulose derivatives, gums, crosslinked polymers and the like; binders such as starch, gelatin, sugars, cellulose derivatives, polyvinyl pyrrolidone and the like; lubricants such as talc, magnesium stearate, colloidal silicon dioxide, polyethylene glycol and mixtures thereof.

It is known to those skilled in the art that an oral controlled drug delivery system should be designed not only with a control on the rate at which it releases the drug over the drug delivery time period (temporal control) but also a control on the location from which it is delivered (spatial control). The spatial control can be achieved by prolonging the period of retention of the system in the stomach. One of the approaches that has been used for achieving spatial control involves increasing the gastric retention of controlled drug delivery systems.

A preferred embodiment of the present invention is a gastric retention controlled drug delivery system. More preferably, the gastric retention controlled drug delivery system of the present invention is capable of floating in the gastric environment. Preferably, the oral controlled release pharmaceutical composition of the present invention is a gastric retention controlled drug delivery system that is retained in the stomach in the fed as well as the fasted state. Still more preferably, the gastric retention controlled drug delivery system comprises metaxalone, a release rate controlling excipient and a gas generating agent.

In accordance with this invention the gastric retention controlled drug delivery system achieves a high degree of swelling in a short time. The release rate controlling excipient used in the present invention may be one or more swellable polymers. The high degree of swelling is achieved by using swellable polymers that provide high and rapid degree of swelling, or by avoiding a high pressure of compaction of the swellable polymers, or by use of highly swellable polymers that inherently compress to a low density.

Examples of swellable polymers that may be used in the present invention include one or more of those listed above. In preferred embodiments the highly swellable polymer is a mixture of a superdisintegrant and one or more binding agents, the binding agent being selected from hydrophilic polymers, preferably highly swellable polymers. In preferred embodiments, the hydrophilic polymer used is a high viscosity cellulose derivative having aqueous solution viscosity ranging from about 500 mPas to about 1,20,000 mPas. A mixture of sodium starch glycolate and high viscosity grade hydroxypropyl methylcellulose is used as the preferred swellable polymer in one embodiment of the present invention.

Sodium starch glycolate is a sodium salt of carboxymethyl ether of starch having a molecular weight in the range of 500,000 to 1,000,000 Daltons, and is commercially available as Explotab® and Primojel®. Sodium starch glycolate causes disintegration by rapid uptake of water, followed by rapid and enormous swelling. The advantage of using sodium starch glycolate as the superdisintegrant is that its effectiveness is not affected by the presence of hydrophobic excipients, such as lubricants, or by increased compression pressure. It is capable of swelling to 300 times its volume in water. Sodium starch glycolate is used as the preferred superdisintegrant in the present invention in an amount ranging from about 5% to about 50% by weight of the composition, preferably from about 10% to about 40% by weight of the composition, more preferably from about 15% to about 30% by weight of the composition.

Hydroxypropyl methylcellulose (HPMC) is a partly O-methylated and O-(2-hydroxypropylated) cellulose, available in different grades that vary in viscosity. The molecular weight of HPMC ranges between 10,000 and 1,500,000. It is commercially available as Benecel MHPC, Methocel and Metolose. The swelling polymer used in the present invention may be HPMC of a particular grade or a mixture of two different grades. In one embodiment of the present invention, a mixture of HPMC K4M grade and HPMC K15M grade is used as the swelling polymer in an amount ranging from about 5% to about 30% by weight of the composition, more preferably from about 10% to about 20% by weight of the composition. The HPMC used also acts as a binding agent.

The oral controlled release pharmaceutical composition of the present invention may include wetting agents selected from the group comprising glycols such as polyethylene glycol of different grades, surfactants such as sodium docusate, polyoxyethylene fatty acid esters, sorbitan fatty acid esters, polyoxyethylene stearates, polyoxyethylene ether, polyoxyethylene-polyoxypropylene copolymers, sodium lauryl sulfate, and mixtures thereof. The wetting agent is used in an amount ranging from about 0.5% to about 5% by weight of the composition.

The gas generating agent used in the gastric retention controlled drug delivery system of the present invention may include a single component that generates gas upon contact with the gastric fluid, or may include a gas generating couple. Gas generating components that may be used in the present invention include carbonates such as calcium carbonate, bicarbonates such as sodium or potassium bicarbonate, sulfites such as sodium sulfite, sodium bisulfite, or sodium metabisulfite, and the like. These salts may be used alone or in combination with an acid source as a gas generating couple. The acid source may be an edible organic acid, a salt of an edible organic acid, or mixtures thereof. Examples of organic acids that may be used include citric acid, malic acid, succinic acid, tartaric acid, fumaric acid, maleic acid, ascorbic acid, glutamic acid, and their salts, and mixtures thereof. The gas generating agent is used in an amount ranging from about 1% to about 50% by weight of the composition, more preferably from about 1% to about 15% by weight of the composition. A mixture of sodium bicarbonate and calcium carbonate is used as the preferred gas generating agent. In yet another embodiment, the gastric retention controlled drug delivery system of the present invention may include a mixture of sodium bicarbonate, calcium carbonate and fumaric acid as the gas generating agent.

The pharmaceutical composition of the present invention may further comprise an excipient that increases the rate of swelling of the delivery system. This excipient may be a water-soluble compound that induces osmosis, or a wicking agent such as microcrystalline cellulose, that promotes the influx of water into the system. Water-soluble compounds suitable for inducing osmosis, i.e. osmotic agents or osmogents, include all pharmaceutically acceptable and pharmacologically inert water-soluble compounds referred to in the pharmacopoeias such as United States Pharmacopoeia, as well as in Remington: The Science and Practice of Pharmacy. Pharmaceutically acceptable water-soluble salts of inorganic or organic acids, or non-ionic organic compounds with high water solubility, e.g. carbohydrates such as sugar, or amino acids, are generally preferred. The examples of agents used for inducing osmosis include inorganic salts such as magnesium chloride or magnesium sulfate, lithium, sodium or potassium chloride, lithium, sodium or potassium hydrogen phosphate, lithium, sodium or potassium dihydrogen phosphate, salts of organic acids such as sodium or potassium acetate, magnesium succinate, sodium benzoate, sodium citrate or sodium ascorbate; carbohydrates such as mannitol, sorbitol, arabinose, ribose, xylose, glucose, fructose, mannose, galactose, sucrose, maltose, lactose, raffinose; water-soluble amino acids such as glycine, leucine, alanine, or methionine; urea and the like, and mixtures thereof. The gastric retention controlled drug delivery system of the present invention may include one or more osmotic agents that increase the rate of swelling of the system. Preferably, the osmotic agent is used in an amount ranging from about 0.5% to about 50% by weight of the composition, more preferably from about 2% to about 40% by weight of the composition. In preferred embodiments the osmotic agent used is mannitol.

The gastric retention controlled drug delivery system of the present invention may also include various pharmaceutically acceptable excipients, for example disintegrants such as starch, cellulose derivatives, gums, crosslinked polymers and the like; binders such as starch, gelatin, sugars, cellulose derivatives, polyvinyl pyrrolidone and the like; lubricants such as talc, magnesium stearate, colloidal silicon dioxide, polyethylene glycol, cellulose derivatives and the like; and mixtures thereof.

Examples of lubricants that may be used in the present invention include talc, magnesium stearate, calcium stearate, aluminum stearate, stearic acid, hydrogenated vegetable oils, colloidal silicon dioxide, polyethylene glycol, cellulose derivatives such as carboxyalkyl cellulose and its alkali salts, or mixtures thereof. In preferred embodiments, the lubricant used is a mixture of polyethylene glycol and magnesium stearate.

The gastric retention controlled drug delivery system of the present invention rapidly swells while maintaining its physical integrity in gastrointestinal fluids for prolonged periods. A low density is achieved by entrapment of the gas generated by the gas generating agent such that the system floats in gastric fluids.

The pharmaceutical composition of the present invention may be obtained by methods known to a person skilled in the art. One embodiment of the present invention comprises the steps of mixing metaxalone with the release rate controlling excipient and other pharmaceutically acceptable excipients and forming a pharmaceutical dosage form by conventional means. In an alternative embodiment, a core may be formed from the mixture of metaxalone and the pharmaceutically acceptable excipients, which may or may not include a release rate controlling excipient; and then the core may be coated by conventional methods with a coating composition comprising the release rate controlling excipient. The pharmaceutical dosage form may be formed by any of the various methods known in the art. It may be formed into capsules by filling the mixture of metaxalone and pharmaceutically acceptable excipients into capsules. Alternatively, the mixture may be formed into granules or pellets by conventional means such as dry granulation, wet granulation, extrusion, spheronisation and the like. The granules or pellets may be filled into capsules or may be compressed into tablets.

The examples that follow are included as illustrations and do not limit the scope of the invention.

### EXAMPLE 1

The gastric retention controlled drug delivery system for metaxalone was prepared as given in Table 1 below—

TABLE 1

| | Quantity | Quantity |
|---|---|---|
| Ingredients | (mg/tablet) | (% w/w) |
| Metaxalone (micronised) | 400.0 | 39.21 |
| Mannitol 25 | 80.0 | 7.84 |
| Hydroxypropyl methylcellulose | 90.0 | 8.82 |
| (HPMC K15M) | | |
| Hydroxypropyl methylcellulose (HPMC K4M) | 55.0 | 5.39 |
| Sodium starch glycolate | 180.0 | 17.65 |
| Sodium bicarbonate | 80.0 | 7.84 |
| Calcium carbonate | 40.0 | 3.92 |
| Povidone K-30 | 15.0 | 1.47 |
| Fumaric acid | 50.0 | 4.90 |
| Sodium lauryl sulphate | 10.0 | 0.98 |
| Polyethylene glycol (PEG 4000) | 10.0 | 0.98 |
| Magnesium stearate | 10.0 | 0.98 |
| Total | 1020.0 | |

Metaxalone, mannitol, HPMC K15M, HPMC K4M, sodium starch glyclolate, sodium bicarbonate and calcium carbonate were sifted and mixed suitably to ensure uniformity. The mixture was granulated using water in a portable PLM model. Wet milling of the mixture was carried out in a multimill using a 10 mm screen. The granules thus obtained were dried (moisture content not more than 3%) and dry milled through a 2 mm screen. The dried granules were then passed through a ASTM (American Society for Testing and Materials) # 16 sieve. A mixture of Povidone K-30, fumaric acid sodium lauryl sulfate, PEG 4000 and magnesium stearate was then used to lubricate the granules. The lubricated mass was finally compressed to obtain the gastric retention controlled drug tablets.

### EXAMPLE 2

The bioavailability of the controlled release metaxalone formulation of the present invention was studied. The gastric retention controlled drug delivery system comprising 400 mg metaxalone (Example 1) was used as the test medication for the same.

The pharmacokinetic assessment was based on the plasma levels of metaxalone measured by blood sampling. Blood samples were obtained before dosing and at the following times after administration of the test medication—0.5, 1, 2, 3, 4, 5, 6, 8, 12, 14, 16 and 24 hours.

Eleven healthy male volunteers were enrolled for the study and all of them completed the study. The subjects were fasted overnight and for 4 hours thereafter. Drinking water was prohibited 2 hours before dosing and 2 hours thereafter, but was allowed ad lib at all other times. Standard meals were provided at 4 hours and 8 hours after dosing and at appropriate times thereafter.

Subjects received the test medication with 240 ml of water at ambient temperature after the overnight fast.

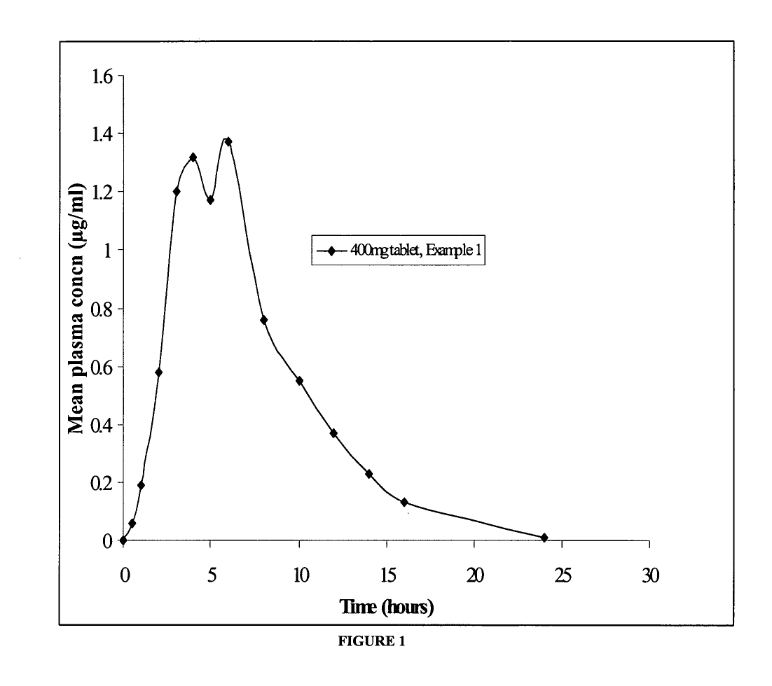

The plasma concentration of metaxalone was determined for samples collected at different time points and averaged over the eleven volunteers. The data is given in Table 3 below. The plasma concentration versus time profile is illustrated in FIG. 1.

TABLE 3

| | Mean Plasma concentration (μg/ml) of |
|---|---|
| Time | metaxalone obtained using controlled |
| (hours) | release tablet (400 mg, Example 1) |
| 0 | 0 |
| 0.5 | 0.06 |
| 1.0 | 0.19 |
| 2.0 | 0.58 |
| 3.0 | 1.20 |
| 4.0 | 1.32 |
| 5.0 | 1.17 |
| 6.0 | 1.37 |
| 8.0 | 0.76 |
| 10.0 | 0.55 |
| 12.0 | 0.37 |
| 14.0 | 0.23 |
| 16.0 | 0.13 |
| 24.0 | 0.01 |

1. An oral controlled release pharmaceutical composition comprising metaxalone, a pharmaceutically acceptable release rate controlling excipient, and pharmaceutically acceptable excipients, wherein the oral controlled release pharmaceutical composition provides peak plasma levels at a time of about 3 hours or more after oral administration of the composition.

2. An oral controlled release pharmaceutical composition as claimed in claim 1, wherein the oral controlled release pharmaceutical composition provides peak plasma levels at about 3 hours to about 8 hours after oral administration of the composition.

3. An oral controlled release pharmaceutical composition as claimed in claim 2 wherein the plasma levels at about 8 hours are at least about 50% of the peak plasma levels.

4. An oral controlled release pharmaceutical composition as claimed in claim 2 wherein the plasma levels from about 3 hours to about 6 hours are at least about 80% of the peak plasma level.

5. An oral controlled release pharmaceutical composition as claimed in claim 2 wherein the peak plasma levels are attained at about 6 hours after administration of the composition.

6. An oral controlled release pharmaceutical composition as claimed in claim 1 wherein the metaxalone is present in an amount of 400 mg.

7. An oral controlled release pharmaceutical composition as claimed in claim 1 wherein the metaxalone is present in an amount of 800 mg.

8. An oral controlled release pharmaceutical composition as claimed in claim 6 wherein the peak plasma levels are more than 1 μg/ml.

9. An oral controlled release pharmaceutical composition as claimed in claim 1 wherein the metaxalone is micronised.

10. An oral controlled release pharmaceutical composition as claimed in claim 1, wherein the pharmaceutically acceptable excipients include wetting agent selected from the group comprising glycols such as polyethylene glycol, surfactants such as sodium docusate, polyoxyethylene fatty acid esters, sorbitan fatty acid esters, polyoxyethylene stearates, polyoxyethylene ether, polyoxyethylene-polyoxypropylene copolymers, sodium lauryl sulfate, and mixtures thereof.

11. An oral controlled release pharmaceutical composition as claimed in claim 1, wherein the pharmaceutically acceptable release rate controlling excipient is a highly swellable polymer.

12. An oral controlled release pharmaceutical composition as claimed in claim 11, wherein the highly swellable polymer is a mixture of a superdisintegrant and a hydrophilic polymer.

13. An oral controlled release pharmaceutical composition as claimed in claim 12, wherein the superdisintegrant used is selected from a group comprising crosslinked polyvinyl pyrrolidone, crosslinked sodium carboxymethyl cellulose and sodium starch glycolate, and the hydrophilic polymer used is a high viscosity cellulose derivative having an aqueous solution viscosity ranging from about 500 mPas to about 1,20,000 mPas for a 2% w/v aqueous solution.

14. An oral controlled release pharmaceutical composition as claimed in claim 13, wherein the sodium starch glycolate is present in an amount ranging from about 10% to about 40% by weight of the composition.

15. An oral controlled release pharmaceutical composition as claimed in claim 13, wherein the high viscosity cellulose derivative is hydroxypropyl methylcellulose having aqueous solution viscosity ranging from 500 mPas to 100,000 mPas for a 2% w/v aqueous solution.

16. An oral controlled release pharmaceutical composition as claimed in claim 15, wherein the hydroxypropyl methylcellulose is present in an amount ranging from about 15% to about 30% by weight of the composition.

17. An oral controlled release pharmaceutical comprising metaxalone, a pharmaceutically acceptable release rate controlling excipient, and pharmaceutically acceptable excipients, wherein the oral controlled release pharmaceutical composition is a gastric retention controlled drug delivery system, wherein the gastric retention controlled drug delivery system provides peak plasma levels at a time of about 3 hours or more after oral administration.

18. A gastric retention controlled drug delivery system as claimed in claim 17, wherein the delivery system provides peak plasma levels at about 3 hours to about 8 hours after oral administration.

19. A gastric retention controlled drug delivery system as claimed in claim 18 wherein the plasma levels at about 8 hours are at least about 50% of the peak plasma levels.

20. A gastric retention controlled drug delivery system as claimed in claim 18 wherein the plasma levels from about 3 hours to about 6 hours are at least about 80% of the peak plasma level.

21. A gastric retention controlled drug delivery system as claimed in claim 18 wherein the peak plasma levels are attained at about 6 hours after oral administration of the gastric retention controlled drug delivery system.

22. A gastric retention controlled drug delivery system as claimed in claim 17 wherein the metaxalone is present in an amount of 400 mg.

23. A gastric retention controlled drug delivery system as claimed in claim 17 wherein the metaxalone is present in an amount of 800 mg.

24. A gastric retention controlled drug delivery system as claimed in claim 22 wherein the peak plasma levels are more than 1 μg/ml.

25. A gastric retention controlled drug delivery system as claimed in claim 17 wherein the metaxalone is micronised.

26. A gastric retention controlled drug delivery system as claimed in claim 17, wherein the pharmaceutically acceptable excipients include wetting agent selected from the group comprising glycols such as polyethylene glycol, surfactants such as sodium docusate, polyoxyethylene fatty acid esters, sorbitan fatty acid esters, polyoxyethylene stearates, polyoxyethylene ether, polyoxyethylene-polyoxypropylene copolymers, sodium lauryl sulfate, and mixtures thereof.

27. A gastric retention controlled drug delivery system as claimed in claim 17, wherein the pharmaceutically acceptable release rate controlling excipient is a highly swellable polymer.

28. A gastric retention controlled drug delivery system as claimed in claim 27, wherein the highly swellable polymer is a mixture of a superdisintegrant and a hydrophilic polymer.

29. A gastric retention controlled drug delivery system as claimed in claim 28, wherein the superdisintegrant used is selected from a group comprising crosslinked polyvinyl pyrrolidone, crosslinked sodium carboxymethyl cellulose and sodium starch glycolate, and the hydrophilic-polymer used is a high viscosity cellulose derivative having an aqueous solution viscosity ranging from about 500 mPas to about 1,20,000 mPas for a 2% w/v aqueous solution.

30. A gastric retention controlled drug delivery system as claimed in claim 29, wherein the sodium starch glycolate is present in an amount ranging from about 10% to about 40% by weight of the composition.

31. A gastric retention controlled drug delivery system as claimed in claim 29, wherein the high viscosity cellulose derivative is hydroxypropyl methylcellulose having aqueous solution viscosity ranging from 500 mPas to 100,000 mPas for a 2% w/v aqueous solution.

32. A gastric retention controlled drug delivery system as claimed in claim 31, wherein the hydroxypropyl methylcellulose is present in an amount ranging from about 15% to about 30% by weight of the composition.

33. A gastric retention controlled drug delivery system as claimed in claim 17 further comprising a gas generating agent.

34. A gastric retention controlled drug delivery system as claimed in claim 33, wherein the gas generating agent is used in an amount ranging from about 1% to about 15% by weight of the composition.

35. A gastric retention controlled drug delivery system as claimed in claim 33, wherein the gas generating agent is selected from a group comprising carbonates, bicarbonates, sulfites and mixtures thereof.

36. A gastric retention controlled drug delivery system as claimed in claim 35, wherein the gas generating agent further comprises an acid source selected from a group comprising organic acids such as citric acid, malic acid, succinic acid, tartaric acid, fumaric acid, maleic acid, ascorbic acid, glutamic acid, or their salts, and mixtures thereof.

37. A gastric retention controlled drug delivery system as claimed in claim 36, wherein the gas generating agent used is a mixture of sodium bicarbonate, calcium carbonate and fumaric acid.

38. A gastric retention controlled drug delivery system as claimed in claim 17, further comprising an osmotic agent.

39. A gastric retention controlled drug delivery system as claimed in claims 38, wherein the osmotic agent is used in an amount ranging from about 2% to about 40% by weight of the composition.

40. A method of relieving pain using the oral controlled release pharmaceutical composition of claim 1.

41. A method of relieving pain using the gastric retention controlled drug delivery system of claim 17.
